SEQUENCE LISTING (20 sequences)

SEQ ID NO: 1 - protein, 121 aa, synthetic construct
  REGION 1..121: note = SARS-CoV-2 ORF8 Amino Acid Sequence
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 2 - protein, 121 aa, synthetic construct
  REGION 1..121: note = RaTG13 ORF8 Amino Acid Sequence
MKLLVFLGILTTVTAFHQECSLQSCAQHQPYVVDDPCPIHFYSKWYIRVGARKSAPLIELCVDEVGSKSPIQYIDIGNYTVSCSPFTINCQEPKLGSLVVRCSFYEDFLEYHDVRVVLDFI

SEQ ID NO: 3 - DNA, 366 bp, synthetic construct
  misc_feature 1..366: note = SARS-CoV-2 ORF8 DNA Sequence
  source 1..366: mol_type = other DNA
atgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaa

SEQ ID NO: 4 - DNA, 366 bp, synthetic construct
  misc_feature 1..366: note = RaTG13 ORF8 DNA Sequence
  source 1..366: mol_type = other DNA
atgaaacttcttgttttcttaggaatcctcacaacagtaactgcatttcaccaagaatgtagtttacagtcatgtgctcaacaccaaccatatgtagttgatgacccgtgtcctattcacttctattctaaatggtacattagagtaggagctagaaaatcagcacctttaattgaattgtgcgtggatgaggttggttctaaatcacccattcagtacatcgatatcggcaattatacagtttcctgttcaccttttacaattaattgccaggagcctaaattgggtagtcttgtagtgcgttgttcgttctatgaagactttttagagtatcatgacgttcgtgttgttttagatttcatctaa

SEQ ID NO: 5 - protein, 8 aa, synthetic construct
  REGION 1..8: note = SARS-CoV-2 ORF8 Conserved Catalytic Core
PFTINCQE

SEQ ID NO: 6 - protein, 5 aa, synthetic construct
  REGION 1..5: note = SARS-CoV ORF8b functional motif
VLVVL

SEQ ID NO: 7 - protein, 8 aa, synthetic construct
  REGION 1..8: note = SARS-CoV-2 ORF8 Fragment
VDEAGSKS

SEQ ID NO: 8 - protein, 7 aa, synthetic construct
  REGION 1..7: note = SARS-CoV-2 ORF8 Fragment
DEAGSKS

SEQ ID NO: 9 - protein, 121 aa, synthetic construct
  REGION 1..121: note = SARS-CoV-2 ORF8 V62L Variant
MKFLVFLGIITTVAAFHQECSLQSCTQHQPYVVDDPCPIHFYSKWYIRVGARKSAPLIELCLDEAGSKSPIQYIDIGNYTVSCLPFTINCQEPKLGSLVVRCSFYEDFLEYHDVRVVLDFI

SEQ ID NO: 10 - protein, 121 aa, synthetic construct
  REGION 1..121: note = SARS-CoV-2 ORF8 L84S Variant
MKFLVFLGIITTVAAFHQECSLQSCTQHQPYVVDDPCPIHFYSKWYIRVGARKSAPLIELCVDEAGSKSPIQYIDIGNYTVSCSPFTINCQEPKLGSLVVRCSFYEDFLEYHDVRVVLDFI

SEQ ID NO: 11 - protein, 121 aa, synthetic construct
  REGION 1..121: note = SARS-CoV-2 ORF8 S24L Variant
MKFLVFLGIITTVAAFHQECSLQLCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 12 - protein, 10 aa, synthetic construct
  REGION 1..10: note = SARS-CoV-2 ORF8 Fragment
LGIITTVAAF

SEQ ID NO: 13 - protein, 10 aa, synthetic construct
  REGION 1..10: note = SARS-CoV-2 ORF8 Fragment
LEYHDVRVVL

SEQ ID NO: 14 - protein, 15 aa, synthetic construct
  REGION 1..15: note = SARS-CoV-2 ORF8 Fragment
SKWYIRVGARKSAPL

SEQ ID NO: 15 - protein, 15 aa, synthetic construct
  REGION 1..15: note = SARS-CoV-2 ORF8 Fragment
HQPYVVDDPCPIHFY

SEQ ID NO: 16 - protein, 15 aa, synthetic construct
  REGION 1..15: note = SARS-CoV-2 ORF8 Fragment
KWYIRVGARKSAPLI

SEQ ID NO: 17 - protein, 15 aa, synthetic construct
  REGION 1..15: note = SARS-CoV-2 ORF8 Fragment
WYIRVGARKSAPLIE

SEQ ID NO: 18 - protein, 15 aa, synthetic construct
  REGION 1..15: note = SARS-CoV-2 ORF8 Fragment
QHQPYVVDDPCPIHF

SEQ ID NO: 19 - protein, 15 aa, synthetic construct
  REGION 1..15: note = SARS-CoV-2 ORF8 Fragment
IHFYSKWYIRVGARK

SEQ ID NO: 20 - protein, 15 aa, synthetic construct
  REGION 1..15: note = SARS-CoV-2 ORF8 Fragment
YIRVGARKSAPLIEL

(19) United States
(12) Patent Application Publication
(10) Pub. No.: US 2024/0261392 A1
(43) Pub. Date: Aug. 8, 2024

(54) COMPOSITIONS AND METHODS TO INCREASE CORONAVIRUS IMMUNE RESPONSE

(71) Applicant: Atossa Therapeutics, Inc., Seattle, WA (US)

(72) Inventor: Steven C. QUAY, Seattle, WA (US)

(21) Appl. No.: 18/568,769

(22) PCT Filed: Jul. 8, 2022
(86) PCT No.: PCT/US2022/036471

§ 371 Date: Dec. 8, 2023

Related U.S. Application Data
(60) Provisional application No. 63/220,389, filed on Jul. 9, 2021.

(51) Int. Cl. A61K 39/215; A61K 39/00; A61K 47/02; A61K 47/10; A61K 47/12; A61K 47/26; A61K 47/40; C12N 7/00; C12N 15/86

(52) U.S. Cl. CPC A61K 39/215; A61K 47/02; A61K 47/10; A61K 47/12; A61K 47/26; A61K 47/40; C12N 7/00; C12N 15/86; A61K 2039/5254; A61K 2039/5256; A61K 2039/53; A61K 2039/54; A61K 2039/545; A61K 2039/55505; A61K 2039/55555; A61K 2039/55561; A61K 2039/55572; C12N 2750/14143; C12N 2770/20034; C12N 2770/20043

(57) ABSTRACT

A composition for increasing an immune response to a coronavirus may contain a coronavirus ORF8 protein, an immunogenic fragment of a coronavirus ORF8 protein, a nucleic acid encoding a coronavirus ORF8 protein, or immunogenic fragment of a coronavirus ORF8 protein. A method for increasing an immune response to a coronavirus may involve administering a composition containing a coronavirus ORF8 protein, an immunogenic fragment of a coronavirus ORF8 protein, a nucleic acid encoding a coronavirus ORF8 protein, or immunogenic fragment of a coronavirus ORF8 protein to a subject. Administration of an ORF8 composition may induce an immune response to the ORF8 protein or immunogenic fragment in the subject and may enhance the immune response of the subject to subsequent a coronavirus infection.

### CROSS-REFERENCE

The present application claims the benefit of U.S. Provisional Application No. 63/220,389, entitled “COMPOSITIONS AND METHODS TO INCREASE CORONAVIRUS IMMUNE RESPONSE,” filed on Jul. 9, 2021, which application is herein incorporated by reference in its entirety for all purposes.

### BACKGROUND

While vaccines targeting the SARS-COV-2 spike protein have been successful in slowing the spread of COVID-19 infections, recent data suggests that immunity conferred by these vaccines may decrease over time. Furthermore, instances of breakthrough infections have been reported, suggesting that the SARS-COV-2 virus may be capable of evading the host immune response. There is a need for vaccines that confer enhanced immunity to SARS-COV-2.

### SUMMARY

In various aspects, the present disclosure provides a composition comprising: a nucleic acid molecule encoding a coronavirus ORF8 peptide, and a lipid particle encapsulating the nucleic acid molecule.

In some aspects, the lipid particle comprises lipids selected from the group consisting of 2[(polyethylene glycol (PEG))-2000]-N,N-ditetradecylacetamide; 1,2-distearoyl-sn-glycero-3-phosphocholine; cholesterol; ((4-hydroxybutyl)azanediyl)bis(hexane-6,1-diyl)bis(2-hexyldecanoate); 1,2-dimyristoyl-rac-glycerol, methoxypolyethylene glycol; heptadecane-9-yl 8-((2-hydroxyethyl) (6-oxo-6-(undecyloxy) hexyl) amino) octanoate; and combinations thereof.

In various aspects, the present disclosure provides a composition comprising: a nucleic acid molecule encoding a coronavirus ORF8 peptide, and a viral vector encapsulating the nucleic acid molecule.

In some aspects, the viral vector is an attenuated viral vector. In some aspects, the viral vector is an adenovirus vector. In some aspects, the adenovirus vector is a replication-deficient adenovirus. In some aspects, the replication-deficient adenovirus is a ChAd0x1 adenovirus, an Ad5 adenovirus, or an Ad26 adenovirus. In some aspects, the viral vector is an attenuated coronavirus.

In some aspects, the nucleic acid molecule is an RNA molecule. In some aspects, the nucleic acid molecule is a DNA molecule. In some aspects, the nucleic acid molecule further encodes an additional coronavirus protein comprising a coronavirus spike protein or an immunogenic fragment thereof, a coronavirus envelope protein or an immunogenic fragment thereof, a coronavirus membrane protein or an immunogenic fragment thereof, a coronavirus nucleocapsid protein or an immunogenic fragment thereof, or combinations thereof. In some aspects, the nucleic acid molecule codes for expression of the coronavirus ORF8 peptide in a subject upon administration of the composition to the subject.

In various aspects, the present disclosure provides a composition comprising: a coronavirus ORF8 peptide, and an adjuvant capable of enhancing an immune response upon administration to a subject.

In some aspects, the adjuvant comprises an aluminum salt, a monophosphorylated lipid A, or a cytosine phosphoguanine. In some aspects, the composition further comprises a carrier protein fused to the coronavirus ORF8 peptide. In some aspects, the carrier protein is adsorbed onto the adjuvant.

In some aspects, the composition further comprises an additional immunogenic peptide. In some aspects, the nucleic acid molecule further encodes an additional immunogenic peptide. In some aspects, the additional immunogenic peptide is an additional coronavirus peptide or an influenza peptide. In some aspects, the additional coronavirus peptide comprises a coronavirus spike protein or an immunogenic fragment thereof, a coronavirus envelope protein or an immunogenic fragment thereof, a coronavirus membrane protein or an immunogenic fragment thereof, or a coronavirus nucleocapsid protein or an immunogenic fragment thereof.

In some aspects, the coronavirus ORF8 peptide comprises an ORF8 protein or an immunogenic fragment thereof. In some aspects, the coronavirus ORF8 peptide comprises a sequence having at least 80% sequence identity to any one of SEQ ID NO: 1 or SEQ ID NO: 9-SEQ ID NO: 11. In some aspects, the coronavirus ORF8 peptide comprises a sequence of any one of SEQ ID NO: 1 or SEQ ID NO: 9-SEQ ID NO: 11. In some aspects, the coronavirus ORF8 peptide comprises from 6 to 120 consecutive amino acid residues of a sequence having at least 80% sequence identity to any one of SEQ ID NO: 1 or SEQ ID NO: 9-SEQ ID NO: 11. In some aspects, the coronavirus ORF8 peptide comprises from 6 to 120 consecutive residues of a sequence of any one of SEQ ID NO: 1 or SEQ ID NO: 9-SEQ ID NO: 11. In some aspects, the coronavirus ORF8 peptide comprises a sequence of any one of SEQ ID NO: 5-SEQ ID NO: 8 or SEQ ID NO: 12-SEQ ID NO: 20.

In some aspects, the composition further comprises a salt, a sugar, a stabilizer, or combinations thereof. In some aspects, the salt comprises sodium phosphate, potassium phosphate, potassium chloride, sodium chloride, sodium acetate, acetic acid, trisodium citrate, citric acid, or combinations thereof. In some aspects, the sugar comprises sucrose, 2-hydroxypropyl-β-cyclodextrin, or combinations thereof. In some aspects, the stabilizer comprises tromethamine, tromethamine hydrochloride, polysorbate-80, ethanol, or combinations thereof. In some aspects, the composition is formulated for intramuscular injection.

In various aspects, the present disclosure provides a method of inducing an immune response in a subject, the method comprising: administering to the subject a composition comprising a nucleic acid molecule encoding a coronavirus ORF8 peptide; delivering the nucleic acid molecule to a cell of the subject; expressing the coronavirus ORF8 peptide in the cell; and inducing an immune response to the coronavirus ORF8 peptide.

In various aspects, the present disclosure provides a method of inducing an immune response in a subject, the method comprising: administering to the subject a composition comprising a coronavirus ORF8 peptide; and inducing an immune response to the coronavirus ORF8 peptide.

In various aspects, the present disclosure provides a method of inducing an immune response in a subject, the method comprising: administering to the subject a composition described herein; and inducing an immune response to the coronavirus ORF8 peptide.

In some aspects, the method further comprises administering an additional coronavirus vaccine to the subject within 90 days of administering the composition. In some aspects, the additional coronavirus vaccine is a SARS-COV vaccine or a SARS-COV-2 vaccine. In some aspects, the method further comprises administering an influenza vaccine to the subject within 90 days of administering the composition. In some aspects, the additional coronavirus vaccine, the influenza vaccine, or both is administered within one day of the composition. In some aspects, the additional coronavirus vaccine, the influenza vaccine, or both is administered within 15 minutes of the composition. In some aspects, the additional coronavirus vaccine, the influenza vaccine, or both is formulated with the composition.

In some aspects, the subject is a previously immunized subject. In some aspects, the subject is a previously infected subject. In some aspects, the subject has partial immunity to the coronavirus. In some aspects, the partial immunity is conferred by a vaccination state or an infection state.

In some aspects, the method further comprises enhancing an immune response to the coronavirus relative to administration of a vaccine lacking the ORF peptide or a vaccine lacking the nucleic acid molecule encoding the ORF8 peptide. In some aspects, the method further comprises reducing the risk of infection by the coronavirus in the subject upon exposure of the subject to the coronavirus. In some aspects, the method further comprises reducing immune invasion of the coronavirus in the subject upon exposure of the subject to the coronavirus. In some aspects, the method further comprises viral entry of the coronavirus in the subject upon exposure of the subject to the coronavirus. In some aspects, the method further comprises reducing viral replication of the coronavirus in the subject upon exposure of the subject to the coronavirus. In some aspects, the method further comprises preventing inhibition of MHC class I by the coronavirus upon exposure of the subject to the coronavirus.

In some aspects, the coronavirus is a sarbecovirus. In some aspects, the coronavirus is SARS-COV or SARS-COV-2. In some aspects, the coronavirus encodes ORF8. In some aspects, the coronavirus inhibits viral antigen presentation in an infected cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the three-dimensional structure of an ORF8 protein from a SARS-CoV-2 coronavirus. The ORF8 protein forms a homodimer connected by a single disulfide bond.

FIG. 2 schematically illustrates a mechanism of SARS-COV-2 immune evasion mediated by ORF8 proteins. ORF8 proteins inhibit host cell immune response to SARS-COV-2 by downregulating major histocompatibility class 1 (MHC-1). Upon infection of a host cell with a virus (e.g., a SARS-COV-2) encoding ORF8 (top), ORF8 protein inhibits MHC-1 mediated viral antigen presentation, preventing surface display of viral epitopes and recognition of the infected cell by host T cells. In contrast, cells infected with a virus (e.g., a SARS-COV-2) with an ORF8 deletion (bottom), viral antigenic peptide epitopes are displayed on the surface of the infected cell and may be recognized by the host T cells, and the infected cell may be killed.

FIG. 3A shows a sequence alignment comparing a RaTG13 coronavirus ORF8 protein (SEQ ID NO: 2, top, “Query”) to a SARS-COV-2 coronavirus ORF8 protein (SEQ ID NO: 1, bottom, “Sbjct”). Amino acids of the SARS-COV-2 coronavirus ORF8 sequence that are identical to the RaTG13 coronavirus ORF8 sequence at the corresponding position are indicated by dots in the subject sequence. Amino acids of the SARS-COV-2 coronavirus ORF8 sequence that differ from the RaTG13 coronavirus ORF8 sequence are indicated by the one-letter amino acid code.

FIG. 3B shows a sequence alignment comparing a RaTG13 coronavirus ORF8 DNA (SEQ ID NO: 4, top, “Query”) to a SARS-COV-2 coronavirus ORF8 DNA (SEQ ID NO: 3, bottom, “Sbjct”). Nucleotides of the SARS-COV-2 coronavirus ORF8 sequence that are identical to the RaTG13 coronavirus ORF8 sequence at the corresponding position are indicated by dots in the subject sequence. Nucleotides of the SARS-COV-2 coronavirus ORF8 sequence that differ from the RaTG13 coronavirus ORF8 sequence are indicated by the one-letter nucleotide code.

### DETAILED DESCRIPTION

The present disclosure provides compositions and methods for increasing an immune response of a subject to a coronavirus (e.g., a SARS-COV-2 coronavirus). SARS-COV-2 is a highly virulent strain of coronavirus that causes COVID-19 which killed more than 6 million people and infected more than 500 million between December of 2019 and July of 2022. SARS-CoV-2 is the seventh coronavirus known to infect humans; SARS-COV, MERS-COV and SARS-CoV-2 can cause severe disease, whereas HKU1, NL63, OC43 and 229E are associated with mild symptoms. The SARS-COV-2 human coronavirus is a 29,903-nucleotide, positive-strand RNA virus that is associated with a variety of highly prevalent and severe diseases, including SARS and Middle East respiratory syndrome (MERS). While the vaccines targeting the SARS-CoV-2 spike protein substantially reduce the risk of infection, data suggests that the immunity conferred by these vaccines may diminish over time and, in some instances, breakthrough infections may occur. Described herein are methods for increasing an immune response of a subject to a coronavirus by inoculating the subject with a composition comprising a coronavirus ORF8 protein, an immunogenic fragment of an ORF8 protein, or a nucleic acid encoding an ORF8 protein or immunogenic fragment of an ORF8 protein.

### Sars-Cov-2 Orf8

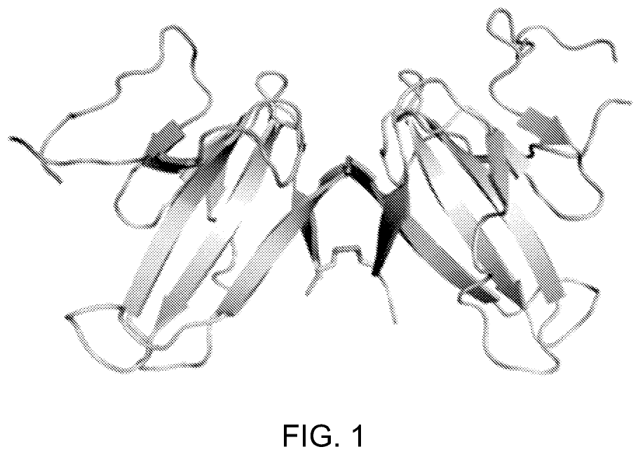

The SARS-COV-2 open reading frame 8 (ORF8) protein is a 121-amino acid protein comprising an N-terminal signal sequence followed by a predicted Ig-like fold. The SARS-COV-2 ORF8 protein forms a homodimer connected by a single disulfide bond. The structure of SARS-COV-2 ORF8 is shown in FIG. 1. Unlike ORF8 found in other coronaviruses, SARS-CoV-2 ORF8 contains solvent solvent-exposed hydrophobic residues that may play a role in SARS-COV-2 pathogenesis. SARS-COV-2 ORF8 contains a histone mimic that disrupts chromatin regulation. ORF8 is predicted to be secreted rather than retained in the ER, and ORF8 antibodies are one of the principal markers of a SARS-COV-2 infection.

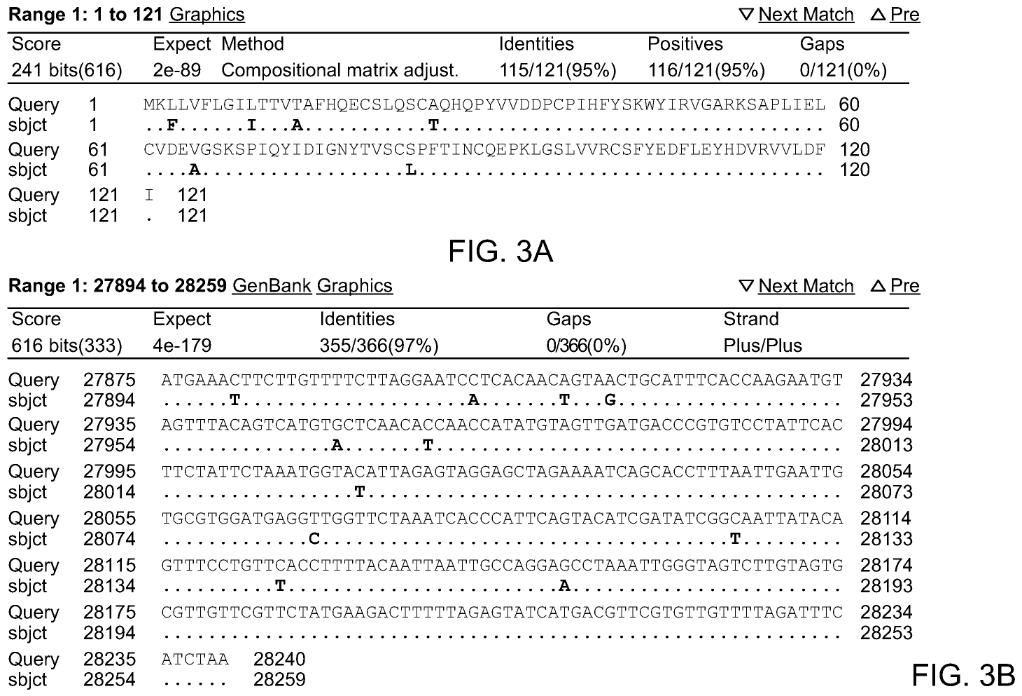

SARS-COV-2 ORF8 has an amino acid sequence of MKFLVFLGIITTVAAFHQECSLQSCTQHQPYVVDDPCPIHFYSKWYIRVGARKSAPLIEL CVDEAGSKSPIQYIDIGNYTVSCLPFTINCQEPKLGSLVVRCSFYEDFLEYHDVRVVLDFI (SEQ ID NO: 1). The SARS-COV-2 ORF8 sequence is highly divergent from other coronavirus ORF8 sequences, sharing less than 20% sequence identity to SARS-COV ORF8. This divergence in the ORF8 sequence may contribute to the increased virulence of SARS-COV-2 relative to other coronaviruses since genetic divergence often contributes to increased virulence in new viral strains. The most conserved region in the ORF8 protein of SARS-COV-2 is PFTINCQE (SEQ ID NO: 5) which is present in the catalytic core of the protein. An amino acid sequence alignment of SARS-COV-2 ORF8 to RaTG13 ORF8, a coronavirus that infects horseshoe bats, is shown in FIG. 3A. The SARS-COV-2 ORF8 (SEQ ID NO: 1) shares 95% amino acid sequence identity to the RaTG13 ORF8 (SEQ ID NO: 2). A nucleic acid sequence alignment of SARS-COV-2 ORF8 to RaTG13 ORF8 is shown in FIG. 3B. The SARS-COV-2 ORF8 (SEQ ID NO: 3) shares 95% nucleic acid sequence identity to the RaTG13 ORF8 (SEQ ID NO: 4).

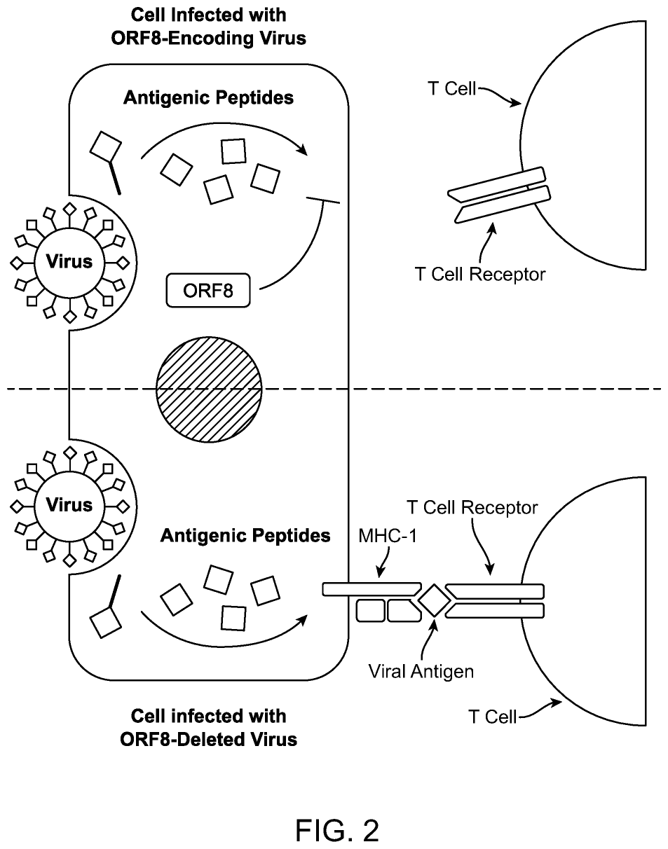

ORF8 is a coronavirus accessory protein may interfere with host inflammatory responses and may contribute to immune evasion of SARS-COV-2, as illustrated in FIG. 2. ORF8 modulates adaptive host immunity and innate immune responses by surpassing interferon-mediated antiviral host responses. SARS-COV ORF8b comprises a functional motif (VLVVL; SEQ ID NO: 6) that may contribute to the induction of host cell stress pathways and activation of macrophages. However, this functional motif is absent from the SARS-COV-2 ORF8 protein, reducing activation of the host cell stress pathways macrophages, and leading to a reduced host immune response. As a result, the symptoms of SARS-COV-2 may be linked to ORF8. The ORF8 protein of SARS-COV-2 mediates immune evasion through down-regulation of major histocompatibility complex (MHC) class I. SARS-COV-2 ORF8 directly interacts with MHC-I molecules and mediates their down-regulation and impair host cell antigen presenting system. Additionally, ORF8 may selectively target MHC-I molecules for lysosomal degradation via autophagy. Thus, SARS-COV-2-infected hosts may be less sensitive to lysis by cytotoxic T lymphocytes.

The ORF8 protein of SARS-COV-2 induces endoplasmic reticulum stress and mediated immune evasion by antagonizing production of interferon β. Both SARS-COV-2 ORF8 genotypes, ORF8L and ORF8S, induced ER stress pathways, antagonize interferon β production, and decrease the nuclear translocation of IRF3 induced by poly. The SARS-COV-2 ORF8 mainly acts as an immune-modulator by down-regulating MHC class I molecules, thereby shielding the infected cells against cytotoxic T cells, killing the target cells. The ORF8 also regulates unfolded protein response (UPR) induced due to the ER stress by triggering the ATF-6 activation, thus enhancing the survivability of infected cells. ORF8 may inhibit induction intracellular aggregation, lysosomal stress, and interleukin-mediated inflammatory responses by activating NLRP3 inflammasomes, thereby inhibiting the host immune response to.

SARS-COV-2 coronaviruses containing an ORF8 deletion are associated with COVID-19 infections with milder symptoms compared to COVID-19 infections caused by SARS-COV-2 coronaviruses with a wild type ORF8. The 382-nucleotide deletion variant (Δ382) causes the deletion of the entire ORF8 protein. Patients with the Δ382 variant exhibit less severe symptoms, including milder hypoxic conditions and low cytokine activity compared to patients infected with the wild type virus. The Δ382 variant shows reduced viral replication ability in human cells, suggesting that SARS-COV-2 ORF8 functions in transmission and viral replication efficiency.

### Compositions for Boosting an Immune Response to a Coronavirus

A composition for boosting an immune response of a subject to a coronavirus (e.g., a SARS-COV-2 coronavirus) may comprise a coronavirus ORF8 protein (e.g., SEQ ID NO: 1), an immunogenic fragment of a coronavirus ORF8 protein (e.g., a fragment of SEQ ID NO: 1 comprising from 6 to 120 amino acid residues), a nucleic acid (e.g., a DNA molecule or an RNA molecule) encoding a coronavirus ORF8 protein, or a nucleic acid encoding a fragment of a coronavirus ORF8 protein. The coronavirus may be SARS-COV, SARS-COV-2, MERS-COV, HKU1, OC43, or 229E. The coronavirus may be a beta-coronavirus.

In some embodiments, the ORF8 protein comprises a sequence of SEQ ID NO: 1. In some embodiments, the ORF8 protein may be an ORF8 variant comprising a sequence having at least 80%, 85%, 90%, 95%, or 99% sequence identity to SEQ ID NO: 1. For example, the ORF8 variant protein may comprise one or more substitutions selected from V62L, L84S, or S24L, relative to SEQ ID NO: 1. In some embodiments, the ORF8 protein comprises a fragment of a sequence having at least 70%, 80%, 85%, 90%, 95%, or 99% sequence identity to SEQ ID NO: 1 or SEQ ID NO: 9-SEQ ID NO: 11. For example, the ORF8 protein fragment may comprise a fragment of an ORF8 protein comprising one or more substitutions selected from V62L, L84S, or S24L, relative to SEQ ID NO: 1. In some embodiments, the ORF8 variant may be an ORF8 protein found in a coronavirus variant (e.g., an ORF8 from a SARS-COV-2 a, B, y, or 8 variant). In some embodiments, the ORF8 variant may be an ORF8 variation found within a coronavirus strain. In some embodiments, an ORF8 variant may be an engineered ORF8. An ORF8 variant may be engineered to have structural homology to wild type ORF8 or to an ORF8 protein found in a coronavirus variant. Structural homology may be determined using bioinformatic software (e.g., ROSETTA, IntFOLD, RaptorX, Biskit, ESyPred3D, Phyre, MODELLER, or the like). Antibodies generated against the engineered ORF8 variant may also recognize and bind wild type ORF8 or an ORF8 from a coronavirus variant. In some embodiments, an ORF8 variant may comprise a V62L substitution (e.g.,
MKFLVFLGIITTVAAFHQECSLQSCTQHQPYVVDDPCPIHFYSKWYIRVGARKSAPLIEL CLDEAGSKSPIQYIDIGNYTVSCLPFTINCQEPKLGSLVVRCSFYEDFLEYHDVRVVLDFI; SEQ ID NO: 9), an L84S substitution (e.g.,
MKFLVFLGIITTVAAFHQECSLQSCTQHQPYVVDDPCPIHFYSKWYIRVGARKSAPLIEL CVDEAGSKSPIQYIDIGNYTVSCSPFTINCQEPKLGSLVVRCSFYEDFLEYHDVRVVLDFI; SEQ ID NO: 10), an S24L substitution (e.g.,
MKFLVFLGIITTVAAFHQECSLQLCTQHQPYVVDDPCPIHFYSKWYIRVGARKSAPLIEL CVDEAGSKSPIQYIDIGNYTVSCLPFTINCQEPKLGSLVVRCSFYEDFLEYHDVRVVLDFI; SEQ ID NO: 11), or combinations thereof. In some embodiments, an ORF8 or ORF8 variant may comprise peptide sequence provided in TABLE 1.

TABLE 1
Exemplary ORF8 Peptides

| Peptide | SEQ ID NO: |
|---|---|
| PFTINCQE | SEQ ID NO: 5 |
| VLVVL | SEQ ID NO: 6 |
| VDEAGSKS | SEQ ID NO: 7 |
| DEAGSKS | SEQ ID NO: 8 |
| LGIITTVAAF | SEQ ID NO: 12 |
| LEYHDVRVVL | SEQ ID NO: 13 |
| SKWYIRVGARKSAPL | SEQ ID NO: 14 |
| HQPYVVDDPCPIHFY | SEQ ID NO: 15 |
| KWYIRVGARKSAPLI | SEQ ID NO: 16 |
| WYIRVGARKSAPLIE | SEQ ID NO: 17 |
| QHQPYVVDDPCPIHF | SEQ ID NO: 18 |
| IHFYSKWYIRVGARK | SEQ ID NO: 19 |
| YIRVGARKSAPLIEL | SEQ ID NO: 20 |

An ORF8 protein fragment may be an immunogenic fragment of an ORF8 protein capable of triggering an immune response against the ORF8 fragment or full-length ORF8 in a subject. Antibodies generated against the ORF8 fragment may also recognize and bind full-length ORF8. An immunogenic fragment of ORF8 may be a surface-exposed fragment of ORF8. An immunogenic fragment of ORF8 may be identified using bioinformatics software (e.g., ExPASy, Protean, or BLASTN) to identify fragments predicted to have high immunogenicity. In some embodiments, an immunogenic fragment may be identified by selecting for fragments of ORF8 that bind to one or more polyclonal antibodies generated against full-length ORF8. In some embodiments, an ORF8 fragment (e.g., an immunogenic fragment) may be an ORF8 truncation found in a coronavirus strain or variant. In some embodiments, an ORF8 fragment may be a fragment shown in TABLE 1.

In some embodiments, an ORF8 fragment may comprise a sequence of VDEAGSKS (SEQ ID NO: 7) or DEAGSKS (SEQ ID NO: 8). An ORF8 fragment may be a fragment of a variant ORF8 sequence having one or more mutations relative to SEQ ID NO: 1. For example, an ORF8 fragment may be a fragment of an ORF8 sequence comprising one or more substitutions selected from V62L, L84S, or S24L relative to SEQ ID NO: 1. In some embodiments, an ORF8 fragment may comprise from 6 to 120 amino acid residues of any one of SEQ ID NO: 1 or SEQ ID NO: 9-SEQ ID NO: 11 or a sequence having at least 70%, 80%, 85%, 90%, 95%, or 99% sequence identity to any one of SEQ ID NO: 1 or SEQ ID NO: 9-SEQ ID NO: 11. The ORF8 fragment may be a surface-exposed fragment of an ORF8 peptide (e.g., a peptide of any one of SEQ ID NO: 1 or SEQ ID NO: 9-SEQ ID NO: 11). In some embodiments, an ORF8 fragment may comprise a sequence of any one of SEQ ID NO: 5-SEQ ID NO: 8 or SEQ ID NO: 12-SEQ ID NO: 20.

In some embodiments, an immunogenic fragment of an ORF8 peptide may have a length of from about 6 to about 20, from about 20 to about 30, from about 30 to about 40, from about 40 to about 50, from about 50 to about 60, from about 60 to about 70, from about 70 to about 80, from about 80 to about 90, from about 90 to about 100, from about 100 to about 110, from about 110 to about 120, from about 6 to about 30, from about 6 to about 50, from about 6 to about 75, from about 6 to about 100, from about 6 to about 120, from about 10 to about 30, from about 10 to about 50, from about 10 to about 75, from about 10 to about 100, from about 10 to about 120, from about 20 to about 50, from about 20 to about 75, from about 20 to about 100, from about 20 to about 120, from about 50 to about 75, from about 50 to about 100, or from about 50 to about 120 amino residues.

In some embodiments, a composition may comprise an ORF8 protein, a fragment of an ORF8 protein, or a nucleic acid encoding an ORF8 protein or protein fragment encapsulated by a delivery vehicle. In some embodiments, the delivery vehicle may comprise a lipid capsid, a viral vector, or an attenuated virus. For example, the delivery vehicle may be a lipid nanoparticle, an adenovirus vector, or an attenuated coronavirus. The delivery vehicle may facilitate delivery of the ORF8 protein, protein fragment, or coding sequence to a host cell.

### Vaccine Compositions

A composition of the present disclosure may be formulated as an ORF8 vaccine for delivery to a subject. An ORF8 vaccine may comprise an ORF8 protein (e.g., any one of SEQ ID NO: 1 or SEQ ID NO: 9-SEQ ID NO: 11) or an immunogenic fragment of an ORF8 protein (e.g., any one of SEQ ID NO: 5-SEQ ID NO: 8 or SEQ ID NO: 12-SEQ ID NO: 20), or an ORF8 vaccine may comprise a nucleic acid encoding for an ORF8 protein (e.g., any one of SEQ ID NO: 1 or SEQ ID NO: 9-SEQ ID NO: 11) or an immunogenic fragment of an ORF8 protein (e.g., any one of SEQ ID NO: 5-SEQ ID NO: 8 or SEQ ID NO: 12-SEQ ID NO: 20). For example, a composition may be formulated as an mRNA vaccine comprising an mRNA encoding an ORF8 protein or protein fragment encapsulated in a lipid particle. In another example, a composition may be formulated an adenovirus vaccine comprising a DNA molecule encoding an ORF8 protein or protein fragment encapsulated in an adenovirus vector. In another example, a composition may be formulated as a subunit vaccine, also referred to as an epitope vaccine, comprising an ORF8 protein or protein fragment.

In some embodiments, an ORF8 vaccine may be formulated in a lipid particle. The ORF8 vaccine may comprise a nucleic acid molecule encoding an ORF8 protein or an immunogenic fragment thereof and a lipid particle encapsulating the nucleic acid molecule, wherein the ORF8 protein is an coronavirus ORF8 protein. The lipid particle may encapsulate a nucleic acid molecule encoding for an ORF8 protein (e.g., any one of SEQ ID NO: 1 or SEQ ID NO: 9-SEQ ID NO: 11) or an immunogenic fragment of an ORF8 protein (e.g., any one of SEQ ID NO: 5-SEQ ID NO: 8 or SEQ ID NO: 12-SEQ ID NO: 20). The nucleic acid molecule may be DNA or RNA (e.g., mRNA). In some embodiments, the lipid particle may comprise lipids selected from the group consisting of 2 [(polyethylene glycol (PEG))-2000]-N,N-ditetradecylacetamide; 1,2-distearoyl-sn-glycero-3-phosphocholine; cholesterol; ((4-hydroxybutyl)azanediyl)bis(hexane-6,1-diyl)bis(2-hexyldecanoate); 1,2-dimyristoyl-rac-glycerol, methoxypolyethylene glycol; heptadecane-9-yl 8-((2-hydroxyethyl) (6-oxo-6-(undecyloxy) hexyl) amino) octanoate; and combinations thereof. For example, the lipid particle may comprise 2[(polyethylene glycol (PEG))-2000]-N,N-ditetradecylacetamide, 1,2-distearoyl-sn-glycero-3-phosphocholine, cholesterol, and ((4-hydroxybutyl)azanediyl)bis(hexane-6,1-diyl)bis(2-hexyldecanoate). In another example, the lipid particle may comprise 1,2-dimyristoyl-rac-glycerol, methoxypolyethylene glycol, 1,2-distearoyl-sn-glycero-3-phosphocholine, cholesterol, and heptadecane-9-yl 8-((2-hydroxyethyl) (6-oxo-6-(undecyloxy) hexyl) amino) octanoate. The ORF8 vaccine comprising a lipid particle encapsulating a nucleic acid molecule may further comprise a salt (e.g., sodium phosphate, potassium phosphate, potassium chloride, sodium chloride, sodium acetate, acetic acid, trisodium citrate, citric acid, or combinations thereof), a sugar (e.g., sucrose, 2-hydroxypropyl-β-cyclodextrin, or combinations thereof), a stabilizer (e.g., tromethamine, tromethamine hydrochloride, polysorbate-80, ethanol, or combinations thereof), or a combination thereof. For example, the ORF8 vaccine may comprise sodium phosphate, potassium phosphate, potassium chloride, sodium chloride, and sucrose. In another example, the ORF8 vaccine may comprise sodium acetate, sucrose, tromethamine, tromethamine hydrochloride, and acetic acid.

In some embodiments, an ORF8 vaccine may be formulated as a viral vector. The ORF8 vaccine may comprise a nucleic acid molecule encoding an ORF8 protein or an immunogenic fragment thereof, and a viral vector encapsulating the nucleic acid molecule, wherein the ORF8 protein is a coronavirus ORF8 protein. The viral vector may encapsulate a nucleic acid molecule encoding for an ORF8 protein (e.g., any one of SEQ ID NO: 1 or SEQ ID NO: 9-SEQ ID NO: 11) or an immunogenic fragment of an ORF8 protein (e.g., any one of SEQ ID NO: 5-SEQ ID NO: 8 or SEQ ID NO: 12-SEQ ID NO: 20). The nucleic acid molecule may be DNA or RNA. The viral vector may be an attenuated virus (e.g., an attenuated coronavirus) or an adenovirus (e.g., a replication-deficient adenovirus). In some embodiments, the adenovirus may be a ChAd0x1 adenovirus, an Ad5 adenovirus, or an Ad26 adenovirus. The ORF8 vaccine comprising a viral vector encapsulating a nucleic acid molecule may further comprise a salt (e.g., sodium phosphate, potassium phosphate, potassium chloride, sodium chloride, sodium acetate, acetic acid, trisodium citrate, citric acid, or combinations thereof), a sugar (e.g., sucrose, 2-hydroxypropyl-β-cyclodextrin, or combinations thereof), a stabilizer (e.g., tromethamine, tromethamine hydrochloride, polysorbate-80, ethanol, or combinations thereof), or a combination thereof. For example, the ORF8 vaccine may comprise polysorbate 80,2-hydroxypropyl-β-cyclodextrin, trisodium citrate, sodium chloride, citric acid, and ethanol.

In some embodiments, an ORF8 vaccine may be formulated as a subunit or epitope vaccine. The ORF8 vaccine may comprise an ORF8 protein or an immunogenic fragment thereof and an adjuvant capable of enhancing an immune response upon administration to a subject, wherein the ORF8 protein is a coronavirus ORF8 protein. The ORF8 vaccine may comprise an ORF8 protein (e.g., any one of SEQ ID NO: 1 or SEQ ID NO: 9-SEQ ID NO: 11) or an immunogenic fragment of an ORF8 protein (e.g., any one of SEQ ID NO: 5-SEQ ID NO: 8 or SEQ ID NO: 12-SEQ ID NO: 20). The adjuvant may comprise an aluminum salt, a monophosphorylated lipid A, or a cytosine phosphoguanine. For example, the adjuvant may be aluminum hydroxide. In some embodiments, the ORF8 vaccine may further comprise a carrier protein fused to the ORF8 protein or the immunogenic fragment. The ORF8 vaccine comprising a viral vector encapsulating a nucleic acid molecule may further comprise a salt (e.g., sodium phosphate, potassium phosphate, potassium chloride, sodium chloride, sodium acetate, acetic acid, trisodium citrate, citric acid, or combinations thereof), a sugar (e.g., sucrose, 2-hydroxypropyl-β-cyclodextrin, or combinations thereof), a stabilizer (e.g., tromethamine, tromethamine hydrochloride, polysorbate-80, ethanol, or combinations thereof), or a combination thereof.

An ORF8 vaccine (e.g., a vaccine comprising an ORF8 peptide or immunogenic fragment or a nucleic acid encoding an ORF8 peptide or immunogenic fragment) may enhance an immune response to a viral infection (e.g., a coronavirus infection) when administered alone or in combination with an additional vaccine (e.g., a coronavirus vaccine or an influenza vaccine). The ORF8 vaccine may induce production of anti-ORF8 antibodies in a subject upon administration of the vaccine. The anti-ORF8 antibodies may facilitate an immune response to a virus (e.g., an ORF8-encoding coronavirus). In some embodiments, the ORF8 vaccine may increase T cell killing of cells infected with an ORF8-encoding virus (e.g., an ORF8-encoding coronavirus). In some embodiments, the ORF8 vaccine may increase T cell killing of cells with inhibited viral antigen presentation (e.g., cells with downregulated MHC-1).

An ORF8 vaccine may be formulated in combination with an additional immunogenic agent, such as a coronavirus spike protein, a fragment of a coronavirus spike protein, a membrane protein or protein fragment, an envelope protein or protein fragment, or a nucleocapsid protein or protein fragment, or a nucleic acid encoding a coronavirus spike protein or spike protein fragment, a membrane protein or protein fragment, an envelope protein or protein fragment, or a nucleocapsid protein or protein fragment. In some embodiments, the composition may be formulated as a supplemental or booster vaccine to boost immunity conferred by a coronavirus vaccine.

### Methods of Boosting an Immune Response to a Coronavirus

A method for boosting an immune response of a subject to a coronavirus (e.g., a SARS-CoV-2 coronavirus) may comprise administering a ORF8 vaccine composition of the present disclosure (e.g., a composition comprising a coronavirus ORF8 protein, an immunogenic fragment of a coronavirus ORF8 protein, or a nucleic acid encoding a coronavirus ORF8 protein or immunogenic fragment of a coronavirus ORF8 protein) to a subject. The coronavirus may be SARS-COV, SARS-COV-2, MERS-COV, HKU1, OC43, or 229E. The coronavirus may be a beta-coronavirus. The coronavirus may be a sarbecovirus. The coronavirus may be an ORF8-encoding coronavirus. The composition may induce an immune response in the subject, producing antibodies against the ORF8 protein. The anti-ORF8 antibodies may facilitate an immune response to a virus (e.g., an ORF8-encoding coronavirus). In some embodiments, the ORF8 vaccine may increase T cell killing of cells infected with an ORF8-encoding virus (e.g., an ORF8-encoding coronavirus). In some embodiments, the ORF8 vaccine may increase T cell killing of cells with inhibited viral antigen presentation (e.g., cells with downregulated MHC-1).

In some embodiments, an ORF8 vaccine composition may be administered as a booster following a coronavirus vaccine (e.g., an mRNA vaccine, an adenovirus vaccine, or a subunit vaccine designed to trigger an immune response to a coronavirus spike protein). In some embodiments, an ORF8 vaccine composition may be formulated as a single vaccine with a coronavirus vaccine. For example, single vaccine formulation may comprise a nucleic acid encoding both an ORF8 protein or protein fragment and a spike protein or spike protein fragment, a membrane protein or protein fragment, an envelope protein or protein fragment, or a nucleocapsid protein or protein fragment. In some embodiments, an ORF8 vaccine composition may be administered concurrently with a coronavirus vaccine. An ORF8 vaccine composition of the present disclosure may be formulated in combination with an additional vaccine or immunogenic agent. For example, the ORF8 vaccine may be formulated in combination with a coronavirus vaccine (e.g., targeting a spike protein) or an influenza vaccine. An ORF8 vaccine composition of the present disclosure may be administered in combination with an additional vaccine. For example, the ORF8 vaccine may administered in combination with a coronavirus vaccine (e.g., targeting a spike protein) or an influenza vaccine. In some embodiments an ORF8 vaccine may be administered as a booster to a previously-vaccinated individual (e.g., an individual who had previously received a coronavirus vaccine) or to a previously-infected individual (e.g., an individual previously infected with a coronavirus).

An ORF8 vaccine may be administered in combination with an additional vaccine (e.g., a coronavirus vaccine or an influenza vaccine). The ORF8 vaccine may be administered within about 6 months, about 5 months, about 4 months, about 3 months, about 90 days, about 60 days, about 30 days, about 15 days, about 2 weeks, about 10 days, about 1 week, about 5 days, about 4 days, about 3 days, about 3 days, about 1 day, about 12 hours, about 6 hours, about 4 hours, about 2 hours, about 1 hour, about 30 minutes, about 15 minutes, about 5 minutes, or about 1 minute of an additional vaccine (e.g., a coronavirus vaccine or an influenza vaccine). In some embodiments, the ORF8 vaccine may be formulated in combination with the additional vaccine.

Administration of an ORF8 vaccine composition may reduce the risk of a coronavirus infection in the subject. In some embodiments, administration of an ORF8 vaccine composition may reduce immune invasion of the coronavirus in the subject. Administration of an ORF8 vaccine composition may reduce viral entry of a coronavirus into a cell of a subject or reduce viral replication of the coronavirus in the subject. In some embodiments, administration of an ORF8 vaccine composition may prevent inhibition of MHC-I. Administration of the composition may enhance immunity of the subject to a coronavirus by inducing production of antibodies against ORF8 in the subject. The antibodies against ORF8 may enhance immunity of the subject by binding to ORF8 produced by an infecting coronavirus and degrading or inhibiting the ORF8, thereby blocking the immune-evading function of ORF8.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

One of ordinary skill will appreciate that the less than (“<”) and greater than (“>”) symbols or terminology used herein can be replaced with less than or equal to (“<”) and greater than or equal to (“>”) symbols, respectively, without departing from the scope of this description.

### EXAMPLES

The invention is further illustrated by the following non-limiting examples.

### Example 1

### Administration of an ORF8 mRNA Vaccine as a Booster to Enhance Immunity to a Coronavirus

This example describes administration of an ORF8 mRNA vaccine as a booster to enhance immunity to a coronavirus. An mRNA encoding an ORF8 protein is encapsulated in a lipid particle. The lipid-encapsulated ORF8 mRNA is formulated as a ORF8 mRNA vaccine for administration to a subject. The subject is a previously-vaccinated subject or a previously-infected subject and already has some level of immunity to the coronavirus. Upon administration of the ORF8 mRNA vaccine to the subject, the ORF8 mRNA enters a cell of the subject and is transcribed into ORF8 protein. The subject generates antibodies against the ORF8 protein, thereby enhancing the immunity of the subject against the coronavirus.

### Example 2

### Administration of a Combination ORF8 mRNA Vaccine to Enhance Immunity to a Coronavirus

This example describes administration of a combination ORF8 mRNA vaccine to enhance immunity to a coronavirus. An mRNA encoding an ORF8 protein and one or more additional coronavirus proteins or protein fragments, such as a coronavirus spike protein, is encapsulated in a lipid particle. The lipid-encapsulated mRNA is formulated as an mRNA vaccine for administration to a subject. Upon administration of the mRNA vaccine to the subject, the mRNA enters a cell of the subject and ORF8 protein is transcribed by the host cell. The subject generates antibodies against the ORF8 protein, thereby enhancing the immunity of the subject against the coronavirus compared to administration of an mRNA vaccine lacking ORF8 mRNA.

### Example 3

### Administration of an ORF8 Adenovirus Vaccine as a Booster to Enhance Immunity to a Coronavirus

This example describes administration of an ORF8 adenovirus vaccine as a booster to enhance immunity to a coronavirus. A DNA encoding an ORF8 protein is encapsulated in an adenovirus vector. The adenovirus-encapsulated ORF8 DNA is formulated as an ORF8 adenovirus vaccine for administration to a subject. The subject is a previously-vaccinated subject or a previously-infected subject and already has some level of immunity to the coronavirus. Upon administration of the ORF8 adenovirus vaccine to the subject, the ORF8 DNA enters a cell of the subject and is transcribed into ORF8 protein. The subject generates antibodies against the ORF8 protein, thereby enhancing the immunity of the subject against the coronavirus.

### Example 4

### Administration of a Combination ORF8 Adenovirus Vaccine to Enhance Immunity to a Coronavirus

This example describes administration of a combination ORF8 adenovirus vaccine to enhance immunity to a coronavirus. A DNA encoding an ORF8 protein and one or more additional coronavirus proteins or protein fragments, such as a coronavirus spike protein, is encapsulated in an adenovirus vector. The adenovirus-encapsulated DNA is formulated as an adenovirus vaccine for administration to a subject. Upon administration of the adenovirus vaccine to the subject, the DNA enters a cell of the subject and ORF8 protein is transcribed by the host cell. The subject generates antibodies against the ORF8 protein, thereby enhancing the immunity of the subject against the coronavirus compared to administration of an adenovirus vaccine lacking ORF8 DNA.

### Example 5

### Administration of an ORF8 Protein Vaccine as a Booster to Enhance Immunity to a Coronavirus

This example describes administration of an ORF8 protein vaccine as a booster to enhance immunity to a coronavirus. An ORF8 protein is formulated as an ORF8 protein vaccine for administration to a subject. The subject is a previously-vaccinated subject or a previously-infected subject and already has some level of immunity to the coronavirus. Upon administration of the ORF8 adenovirus vaccine to the subject, the subject generates antibodies against the ORF8 protein, thereby enhancing the immunity of the subject against the coronavirus.

### Example 6

### Administration of a Combination ORF8 Protein Vaccine to Enhance Immunity to a Coronavirus

This example describes administration of a combination ORF8 protein vaccine to enhance immunity to a coronavirus. An ORF8 protein and one or more additional coronavirus proteins or protein fragments, such as a coronavirus spike protein, are formulated as a protein vaccine for administration to a subject. Upon administration of the adenovirus vaccine to the subject, the subject generates antibodies against the ORF8 protein, thereby enhancing the immunity of the subject against the coronavirus compared to administration of a protein vaccine lacking ORF8 DNA.

### Example 7

### Formulation of a Lipid Particle ORF8 Vaccine

This example describes formulation of a lipid particle ORF8 vaccine. An RNA molecule encoding an ORF8 peptide (e.g., an ORF8 protein of any one of SEQ ID NO: 1 or SEQ ID NO: 9-SEQ ID NO: 11 or an immunogenic fragment of any one of SEQ ID NO: 5-SEQ ID NO: 8 or SEQ ID NO: 12-SEQ ID NO: 20 is encapsulated in a lipid particle. Optionally, the RNA molecule further encodes a coronavirus spike protein or an immunogenic fragment thereof, a coronavirus envelope protein or an immunogenic fragment thereof, a coronavirus membrane protein or an immunogenic fragment thereof, a coronavirus nucleocapsid protein or an immunogenic fragment thereof. The lipid particle comprises 2[(polyethylene glycol (PEG))-2000]-N,N-ditetradecylacetamide, 1,2-distearoyl-sn-glycero-3-phosphocholine, cholesterol, and ((4-hydroxybutyl)azanediyl)bis(hexane-6,1-diyl)bis(2-hexyldecanoate). The vaccine formulation further comprises sodium phosphate, potassium phosphate, potassium chloride, sodium chloride, and sucrose. The ORF8 vaccine is formulated for intramuscular administration.

### Example 8

### Additional Formulation of a Lipid Particle ORF8 Vaccine

This example describes an additional formulation of a lipid particle ORF8 vaccine. An RNA molecule encoding an ORF8 peptide (e.g., an ORF8 protein of any one of SEQ ID NO: 1 or SEQ ID NO: 9-SEQ ID NO: 11 or an immunogenic fragment of any one of SEQ ID NO: 5-SEQ ID NO: 8 or SEQ ID NO: 12-SEQ ID NO: 20 is encapsulated in a lipid particle. Optionally, the RNA molecule further encodes a coronavirus spike protein or an immunogenic fragment thereof, a coronavirus envelope protein or an immunogenic fragment thereof, a coronavirus membrane protein or an immunogenic fragment thereof, a coronavirus nucleocapsid protein or an immunogenic fragment thereof. The lipid particle comprises 1,2-dimyristoyl-rac-glycerol, methoxypolyethylene glycol, 1,2-distearoyl-sn-glycero-3-phosphocholine, cholesterol, and heptadecane-9-yl 8-((2-hydroxyethyl) (6-oxo-6-(undecyloxy) hexyl) amino) octanoate. The vaccine formulation further comprises sodium acetate, sucrose, tromethamine, tromethamine hydrochloride, and acetic acid. The ORF8 vaccine is formulated for intramuscular administration.

### Example 9

### Formulation of an Adenovirus ORF8 Vaccine

This example describes formulation of an adenovirus ORF8 vaccine. A DNA molecule encoding an ORF8 peptide (e.g., an ORF8 protein of any one of SEQ ID NO: 1 or SEQ ID NO: 9-SEQ ID NO: 11 or an immunogenic fragment of any one of SEQ ID NO: 5-SEQ ID NO: 8 or SEQ ID NO: 12-SEQ ID NO: 20 is encapsulated in an adenovirus vector. Optionally, the DNA molecule further encodes a coronavirus spike protein or an immunogenic fragment thereof, a coronavirus envelope protein or an immunogenic fragment thereof, a coronavirus membrane protein or an immunogenic fragment thereof, a coronavirus nucleocapsid protein or an immunogenic fragment thereof. The adenovirus is an Ad26 adenovirus. The vaccine formulation further comprises polysorbate 80,2-hydroxypropyl-β-cyclodextrin, trisodium citrate, sodium chloride, citric acid, and ethanol. The ORF8 vaccine is formulated for intramuscular administration.

### Example 10

### Formulation of an Epitope ORF8 Vaccine

This example describes formulation of an epitope ORF8 vaccine. The vaccine comprises an ORF8 peptide (e.g., an ORF8 protein of any one of SEQ ID NO: 1 or SEQ ID NO: 9-SEQ ID NO: 11 or an immunogenic fragment of any one of SEQ ID NO: 5-SEQ ID NO: 8 or SEQ ID NO: 12-SEQ ID NO: 20 fused to a carrier protein. Optionally, the vaccine further comprises a coronavirus spike protein or an immunogenic fragment thereof, a coronavirus envelope protein or an immunogenic fragment thereof, a coronavirus membrane protein or an immunogenic fragment thereof, a coronavirus nucleocapsid protein or an immunogenic fragment thereof fused to a carrier protein. The ORF8 peptide fused to the carrier protein is adsorbed to an aluminum hydroxide adjuvant. The vaccine formulation further comprises sodium chloride. The ORF8 vaccine is formulated for intramuscular administration.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
a nucleic acid molecule encoding a coronavirus ORF8 peptide, and
a lipid particle encapsulating the nucleic acid molecule.

2. The composition of claim 1, wherein the lipid particle comprises lipids selected from the group consisting of 2[(polyethylene glycol (PEG))-2000]-N,N-ditetradecylacetamide; 1,2-distearoyl-sn-glycero-3-phosphocholine; cholesterol; ((4-hydroxybutyl)azanediyl)bis(hexane-6,1-diyl)bis(2-hexyldecanoate); 1,2-dimyristoyl-rac-glycerol, methoxypolyethylene glycol; heptadecane-9-yl 8-((2-hydroxyethyl) (6-oxo-6-(undecyloxy) hexyl) amino) octanoate; and combinations thereof.

3. A composition comprising:
a nucleic acid molecule encoding a coronavirus ORF8 peptide, and
a viral vector encapsulating the nucleic acid molecule.

4. The composition of claim 3, wherein the viral vector is an attenuated viral vector.

5. The composition of claim 3 or claim 4, wherein the viral vector is an adenovirus vector.

6. The composition of claim 5, wherein the adenovirus vector is a replication-deficient adenovirus.

7. The composition of claim 6, wherein the replication-deficient adenovirus is a ChAd0x1 adenovirus, an Ad5 adenovirus, or an Ad26 adenovirus.

8. The composition of claim 3 or claim 4, wherein the viral vector is an attenuated coronavirus.

9. The composition of any one of claims 1-8, wherein the nucleic acid molecule is an RNA molecule.

10. The composition of any one of claims 1-8, wherein the nucleic acid molecule is a DNA molecule.

11. The composition of any one of claims 1-10, wherein the nucleic acid molecule further encodes an additional coronavirus protein comprising a coronavirus spike protein or an immunogenic fragment thereof, a coronavirus envelope protein or an immunogenic fragment thereof, a coronavirus membrane protein or an immunogenic fragment thereof, a coronavirus nucleocapsid protein or an immunogenic fragment thereof, or combinations thereof.

12. The composition of any one of claims 1-11, wherein the nucleic acid molecule codes for expression of the coronavirus ORF8 peptide in a subject upon administration of the composition to the subject.

13. A composition comprising:
a coronavirus ORF8 peptide, and
an adjuvant capable of enhancing an immune response upon administration to a subject.

14. The composition of claim 13, wherein the adjuvant comprises an aluminum salt, a monophosphorylated lipid A, or a cytosine phosphoguanine.

15. The composition of claim 13 or claim 14, further comprising a carrier protein fused to the coronavirus ORF8 peptide.

16. The composition of claim 15, wherein the carrier protein is adsorbed onto the adjuvant.

17. The composition of any one of claims 13-16, wherein the composition further comprises an additional immunogenic peptide.

18. The composition of any one of claims 1-12, wherein the nucleic acid molecule further encodes an additional immunogenic peptide.

19. The composition of claim 17 or claim 18, wherein the additional immunogenic peptide is an additional coronavirus peptide or an influenza peptide.

20. The composition of claim 19, wherein the additional coronavirus peptide comprises a coronavirus spike protein or an immunogenic fragment thereof, a coronavirus envelope protein or an immunogenic fragment thereof, a coronavirus membrane protein or an immunogenic fragment thereof, or a coronavirus nucleocapsid protein or an immunogenic fragment thereof.

21. The composition of any one of claims 1-20, wherein the coronavirus ORF8 peptide comprises an ORF8 protein or an immunogenic fragment thereof.

22. The composition of any one of claims 1-21, wherein the coronavirus ORF8 peptide comprises a sequence having at least 80% sequence identity to any one of SEQ ID NO: 1 or SEQ ID NO: 9-SEQ ID NO: 11.

23. The composition of any one of claims 1-22, wherein the coronavirus ORF8 peptide comprises a sequence of any one of SEQ ID NO: 1 or SEQ ID NO: 9-SEQ ID NO: 11.

24. The composition of any one of claims 1-23, wherein the coronavirus ORF8 peptide comprises from 6 to 120 consecutive amino acid residues of a sequence having at least 80% sequence identity to any one of SEQ ID NO: 1 or SEQ ID NO: 9-SEQ ID NO: 11.

25. The composition of any one of claims 1-24, wherein the coronavirus ORF8 peptide comprises from 6 to 120 consecutive residues of a sequence of any one of SEQ ID NO: 1 or SEQ ID NO: 9-SEQ ID NO: 11.

26. The composition of any one of claims 1-25, wherein the coronavirus ORF8 peptide comprises a sequence of any one of SEQ ID NO: 5-SEQ ID NO: 8 or SEQ ID NO: 12-SEQ ID NO: 20.

27. The composition of any one of claims 1-26, further comprising a salt, a sugar, a stabilizer, or combinations thereof.

28. The composition of claim 27, wherein the salt comprises sodium phosphate, potassium phosphate, potassium chloride, sodium chloride, sodium acetate, acetic acid, trisodium citrate, citric acid, or combinations thereof.

29. The composition of claim 27 or claim 28, wherein the sugar comprises sucrose, 2-hydroxypropyl-β-cyclodextrin, or combinations thereof.

30. The composition of any one of claims 27-29, wherein the stabilizer comprises tromethamine, tromethamine hydrochloride, polysorbate-80, ethanol, or combinations thereof.

31. The composition of any one of claims 1-30, wherein the composition is formulated for intramuscular injection.

32. A method of inducing an immune response in a subject, the method comprising:
administering to the subject a composition comprising a nucleic acid molecule encoding a coronavirus ORF8 peptide;
delivering the nucleic acid molecule to a cell of the subject;
expressing the coronavirus ORF8 peptide in the cell; and
inducing an immune response to the coronavirus ORF8 peptide.

33. A method of inducing an immune response in a subject, the method comprising:
administering to the subject a composition comprising a coronavirus ORF8 peptide; and
inducing an immune response to the coronavirus ORF8 peptide.

34. A method of inducing an immune response in a subject, the method comprising:
administering to the subject the composition of any one of claims 1-31; and
inducing an immune response to the coronavirus ORF8 peptide.

35. The method of any one of claims 32-34, further comprising administering an additional coronavirus vaccine to the subject within 90 days of administering the composition.

36. The method of claim 35, wherein the additional coronavirus vaccine is a SARS-COV vaccine or a SARS-COV-2 vaccine.

37. The method of any one of claims 32-36, further comprising administering an influenza vaccine to the subject within 90 days of administering the composition.

38. The method of any one of claims 35-37, wherein the additional coronavirus vaccine, the influenza vaccine, or both is administered within one day of the composition.

39. The method of any one of claims 35-38, wherein the additional coronavirus vaccine, the influenza vaccine, or both is administered within 15 minutes of the composition.

40. The method of any one of claims 35-39, wherein the additional coronavirus vaccine, the influenza vaccine, or both is formulated with the composition.

41. The method of any one of claims 35-40, wherein the subject is a previously immunized subject.

42. The method of any one of claims 35-41, wherein the subject is a previously infected subject.

43. The method of any one of claims 35-42, wherein the subject has partial immunity to the coronavirus.

44. The method of claim 43, wherein the partial immunity is conferred by a vaccination state or an infection state.

45. The method of any one of claims 32-44, further comprising enhancing an immune response to the coronavirus relative to administration of a vaccine lacking the ORF peptide or a vaccine lacking the nucleic acid molecule encoding the ORF8 peptide.

46. The method of any one of claims 32-45, further comprising reducing the risk of infection by the coronavirus in the subject upon exposure of the subject to the coronavirus.

47. The method of any one of claims 32-46, further comprising reducing immune invasion of the coronavirus in the subject upon exposure of the subject to the coronavirus.

48. The method of any one of claims 32-47, further comprising viral entry of the coronavirus in the subject upon exposure of the subject to the coronavirus.

49. The method of any one of claims 32-48, further comprising reducing viral replication of the coronavirus in the subject upon exposure of the subject to the coronavirus.

50. The method of any one of claims 32-49, further comprising preventing inhibition of MHC class I by the coronavirus upon exposure of the subject to the coronavirus.

51. The method of any one of claims 32-50, wherein the coronavirus is a sarbecovirus.

52. The method of any one of claims 32-51, wherein the coronavirus is SARS-COV or SARS-COV-2.

53. The method of any one of claims 32-52, wherein the coronavirus encodes ORF8.

54. The method of any one of claims 32-53, wherein the coronavirus inhibits viral antigen presentation in an infected cell.
